(19) United States
(12) Patent Application Publication
(10) Pub. No.: US 2005/0239145 A1
(43) Pub. Date: Oct. 27, 2005

(54) Flow-through membrane assays for carbohydrates using labeled lectins

(76) Inventor: Albert Chu, Hillsborough, CA (US)

Correspondence Address: DORSEY & WHITNEY LLP, 555 CALIFORNIA STREET, SUITE 1000, SUITE 1000, SAN FRANCISCO, CA, 94104, US

(21) Appl. No.: 11/014,627

(22) Filed: Dec. 15, 2004

Related U.S. Application Data
(63) Continuation of application No. 10/023,205, filed on Dec. 14, 2001, now abandoned.
(60) Provisional application No. 60/256,143, filed on Dec. 15, 2000.

(51) Int. Cl. G01N 33/53

(52) U.S. Cl. 435/7.5

(57) ABSTRACT

The present invention comprises a method for rapid detection of at least a first carbohydrate in a carbohydrate-containing sample molecule comprising the steps of: (a) retaining the sample molecule on a region of one liquid permeable reaction membrane, (b) flowing a solution of first lectin, capable of binding the first carbohydrate, through the one reaction membrane to bind the first lectin to the retained first carbohydrate, the lectin being directly conjugated to a label prior to binding to the carbohydrate or being bound to a labeled separate molecule only after the first lectin has been bound to the carbohydrate, and (c) thereafter, detecting the label bound on the one reaction membrane, indicating the presence of the first carbohydrate.

### BACKGROUND OF THE INVENTION

The technical field of this invention concerns methods for determining the presence of carbohydrates on sample glycoproteins using labeled lectins. Glycoproteins have been analyzed by a variety of techniques, some of which use labeled lectins, which specifically bind for carbohydrates. A discussion of lectins, their carbohydrates and antibody specificities and their use in conjugated form, such as colloidal gold-labeled lectins, are disclosed in a current publication by EY Laboratories, Inc. entitled “Lectins Lectin Conjugates” (2000) (herein, “the EY publication”). However, the assay techniques known for such carbohydrate analyses, even ones including labeled lectins, are time-consuming and expensive.

Various types of analytical devices, and methods employing the devices, have been used for immunoassays. Many of these devices employ reaction membranes onto which a receptor, capable of specifically binding to the target substance, is immobilized. In the assay that employs these types of devices, typically the sample to be tested is applied to the reaction membrane. If target substance is present in the sample, it binds to the immobilized receptor. Various methods are used to determine whether the target substance has bound to the receptor, thus indicating its presence in the sample. For immunoassays, where the target substance is an antigen, it is common to use antibodies that are capable of specifically binding to the antigen and that are labeled with detectable markers. When the labeled antibody is added to the reaction membrane, it will bind to the target antigen, if present, and the marker (e.g. fluorescent label, colored reagent, detectable enzyme marker, etc.) is detected.

Membrane-based analytical assays and devices have greatly simplified medical diagnostics. The results of a membrane-based analytical assay can be obtained in a matter of minutes. Quantitative results can be provided by special instruments designed to read the test results. Various types of other devices for flow-through membrane-based immunoassays are described in U.S. Pat. No. 5,006,464 to Chu et al., U.S. Pat. No. 4,818,677 to Hay-Kaufman et al., and U.S. Pat. No. 4,632,901 to Valkirs et al., and U.S. Pat. No. 5,185,127 to Vonk et al. However, such devices and methods have not been used for carbohydrate analysis.

Rapid test kits which can be performed in a few minutes or less are sold by EY Laboratories, Inc. under the trademark InstantChek™. These typically use immunological sandwich assays. Such test kits are described in U.S. Pat. No. 5,885,626, incorporated herein by reference.

### SUMMARY OF THE INVENTION

The present invention comprises a method for rapid detection of at least a first carbohydrate in a carbohydrate-containing sample molecule comprising the steps of:
(a) retaining the sample molecule on a region of one liquid permeable reaction membrane,
(b) flowing a solution of first lectin, capable of binding the first carbohydrate, through the one reaction membrane to bind the first lectin to the retained first carbohydrate, the lectin being directly conjugated to a label prior to binding to the carbohydrate or being bound to a labeled separate molecule only after the first lectin has been bound to the carbohydrate, and
(c) thereafter, detecting the label bound on the one reaction membrane, indicating the presence of the first carbohydrate.

### BRIEF DESCRIPTION OF THE DRAWINGS

FIG. 1 is an exploded view of an analytical assay device that can be used in the practice of the invention.

FIG. 2 is a top view of an assembled analytical assay device that can be used in the practice of the invention.

FIG. 3 is a cross-section elevation view of the layers of the analytical device of FIG. 2 along the plane 3-3.

FIG. 4a to 4f show that the configuration of the receptor area(s) and the shape of the reaction membrane can vary.

FIG. 5 illustrates a dose response curve according to the present invention.

FIG. 6 illustrates a pH profile for use in the present invention.

### DETAILED DESCRIPTION OF THE INVENTION

In the present method one or more carbohydrates in a sample are detected using a rapid assay of the general type illustrated in U.S. Pat. No. 6,284,194 (used for immunoassays) and as described in the membrane-based prior art discussed in the Background of the Invention. Such carbohydrate group-containing or—linked sample molecules are termed “glycoconjugates,” and include glycoproteins, neoglycoproteins, monosaccarides, di- and tri-oligosaccharides and oligosaccharides. For simplicity of description, glycoprotein sample molecules will be referred to as the glyco conjugates unless otherwise specified.

In one embodiment, the sample is directly retained on a region of the liquid permeable reaction membrane of such a test kit. A solution of the labeled lectin, capable of binding carbohydrate on the sample, flows through the reaction membrane to selectively bind the lectin to the carbohydrate. The surface is typically washed by flowing a washing solution through the reaction membrane. Then, the labeled lectin bound to the membrane can be detected and quantitatively analyzed by comparison to a known carbohydrate standard curve of the amount of the carbohydrate. For relatively pure samples, the washing step may be eliminated, particularly for a qualitative assay. As discussed hereinafter, in one embodiment, the glycoprotein is deposited directly on the membrane and the labeled lectin reacts with that deposited carbohydrate. In a preferred method, a sandwich assay is used. Specifically, an immobilized receptor capable of binding the sample is deposited in a limited region of the reaction membrane. The sample is retained on the reaction membrane by binding to the immobilized receptor. Then, the bound carbohydrate reacts with the labeled lectin. Thereafter the reaction membrane is washed and the labeled lectin is detected as an indication of the carbohydrate.

The immobilized receptor may comprise a second lectin of the same or different type from the labeled lectin in a sandwich assay. With a lectin of a different type, each lectin can bind to a specific carbohydrate, providing more information in the analysis. However, it is important to select the two lectins to avoid cross-reactivity which could create a false positive.

As used herein, the term “lectin” is used to refer broadly to a protein or glycoprotein that binds to a carbohydrate. Thus, it encompasses the current broad scope of that term typically derived from natural animal and plant sources, and which bind carbohydrates by affinity. Animal sources include vertebrate or invertebrate animals, e.g., snails, fish or the like. Plant sources include seeds and bark. Examples of such lectins are set forth in the EY publication. In addition, the term “lectin” herein encompasses glycoproteins and proteins not normally termed lectins, which immunologically bind carbohydrates, such as antibodies, e.g., monoclonal antibodies. Examples of such antibodies are set forth in the current Seikagaku American catalog.

The present invention provides a particularly effective way to selectively detect different carbohydrates in the sample. In one embodiment, referred to herein as the “checker board approach”, multiple reaction surfaces separated from each other are provided in separate membrane which are either test kits bound together or separated. As used herein, the term “membrane test kit” or “membrane based assay” type of tests described in U.S. Pat. No. 5,885,626 in which sample is retained on a membrane and labeled reagents flow through the membrane, part of which binds to the sample. Preferably, each square in the checkerboard is a membrane test kit. The sample is retained in a limited region of each of the reaction membranes. The assay is repeated using at least a first and a second labeled lectin of a different type from the labeled first lectin. The second lectin is capable of selectively binding the second carbohydrate on the sample. In this manner, the positive or negative results using the labeled first and second lectin which are selective for specific carbohydrates can provide quantitative information regarding two different carbohydrates in the glycoprotein sample. As will be described hereinafter, this checkerboard approach can be expanded using as many different lectins which are selective for carbohydrates to quantitatively analyze the carbohydrate on the glycoprotein sample. It is important to have detailed information on the reactivities of each lectin for each carbohydrate and cross-reactivity for different lectins used in the assay under reaction conditions. Then, the unknown samples may be analyzed.

In contrast to prior art techniques, the present invention permits a rapid quantitative assay for carbohydrates in which a carbohydrate can be analyzed in less than 1 hour to less than 5 minutes, more preferably less than 2 minutes, or even in 1 minute or less. This is based in part on the recognition that specific lectins bind selectively to some but not all carbohydrates (e.g., monosaccarides, such as mannose, GleNAc, gelatose, a-fructose or sialic acid) in different degrees. Using the present assays, the information obtained from the binding or lack thereof of specific lectins to the carbohydrates identify the molecules.

One of the advantages of this system is the ability to rapidly detect a large number of different carbohydrates in the glycoprotein sample by using the well-known binding specificities of different lectins for their corresponding carbohydrates. A summary of some of these binding specificities is set forth on the back page of the EY publication. Other background information on the analysis of glycoproteins using lectins is illustrated in Glycoanalysis Protocols, 2nd Edition (edited by Elizabeth F. Hounsel) and in Lectin Methods and Protocols (edited by Jonathan M. Rhodes and Jeremy D. Milton), Carbohydrate Biotechnology Protocols (edited by Christopher Buck).

Some parameters which can affect the assay include the pH levels, buffer species, concentrations of reagents, ionic strength of the buffer, particularly divalent ions such as calcium or magnesium, and concentration of the glycoprotein or other sample molecules.

The relative ion strength of the solution in which the lectin to the carbohydrate can be an important parameter in the method. Preferably, some of the lectin would interact better with carbohydrate in a higher ionic strength working environment.

Suitable buffer species include conventional ones such as sodium phosphate. Tris glycine buffer, Tris HCl buffer, or borate buffer. If calcium is used as a cation in a phosphate buffer, the calcium concentration should be below a level at which the phosphate precipitates, suitably concentration less than one mM in concentration. Sodium chloride can be used to increase the ionic strength of the buffer. In some instances a high salt concentration may cause background. Calcium serves as a cofactor to assist binding of lectins to carbohydrates. Suitable concentrators are from 0.05 mM to 5 mM.

Suitable buffer concentrations can vary from about 20 mM to 200 mM, preferably from about 10-100 mM. The buffer serves to solubilize the lectin and glycoprotein to be deposited or inoculated on the membrane. A borate buffer (at a 10 mM or higher) with 0.02% sodium azide as preservative is a particularly effective buffer for solubilizing the glycoprotein.

The total ionic strength of the buffer used during reaction of the lectin and carbohydrate forces the molecules together to enhance complex formation and stabilize the complex. The ionic strength will depend on the binding constant of the lectin and carbohydrate. Total ionic strength may vary from about 0.02M to 0.7M.

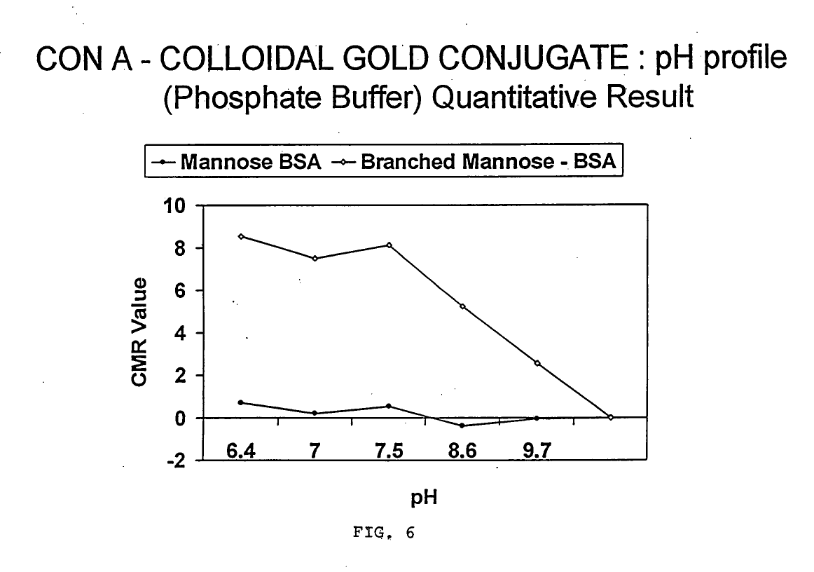

Another parameter of potential significance is pH. Typically the pH range is between about 5.5 and 10, preferably from about 6.0 to 8.5 depending on the carbohydrate and lectin. In FIG. 6, described hereinafter, one example of the relationship of the pH profile of Con A to carbohydrate binding is illustrated. FIG. 6 suggests performing the assay at a pH not to exceed 8.

Various configurations of an analytical assay device can be used in the practice of the invention. Suitable devices are already known in the art and are disclosed in U.S. Pat. No. 5,006,464 to Chu et al., U.S. Pat. No. 4,818,677 to Hay-Kaufman et al., and U.S. Pat. No. 4,632,901 to Valkirs et al. The device disclosed U.S. Pat. No. 5,885,526 is a preferred device for use in the practice of the invention because it is relatively inexpensive and easy to manufacture in comparison with other analytical assay devices.

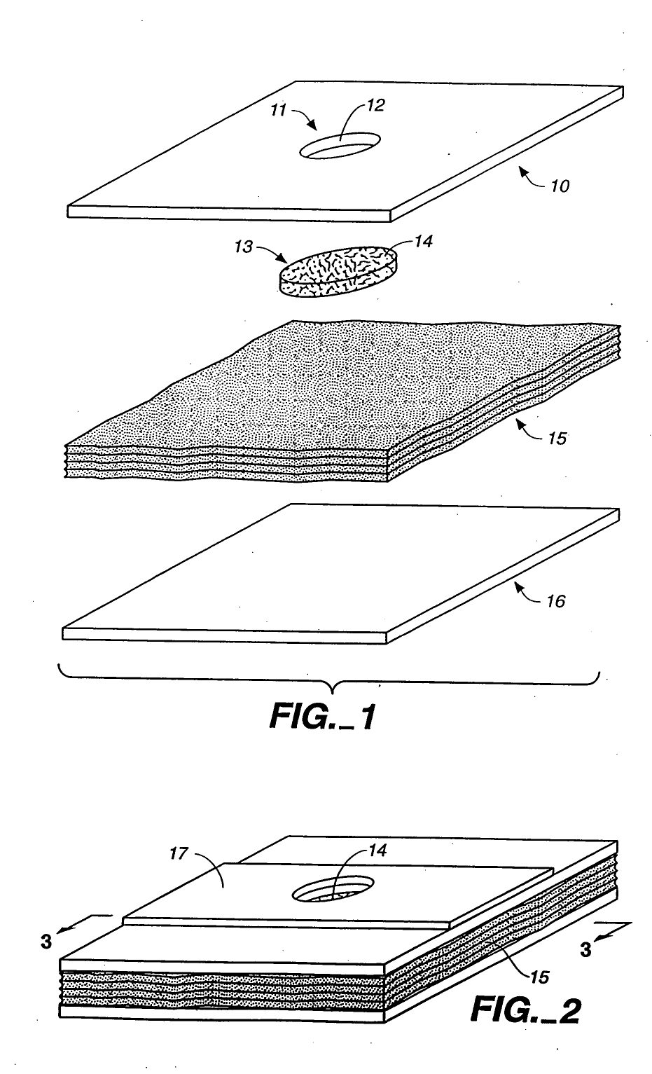

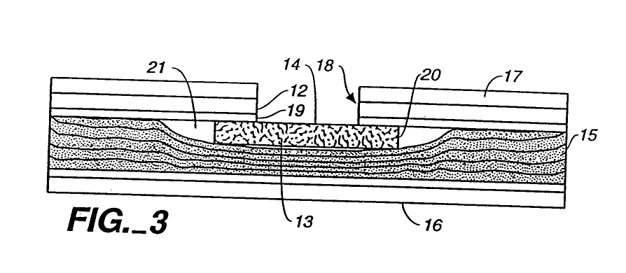

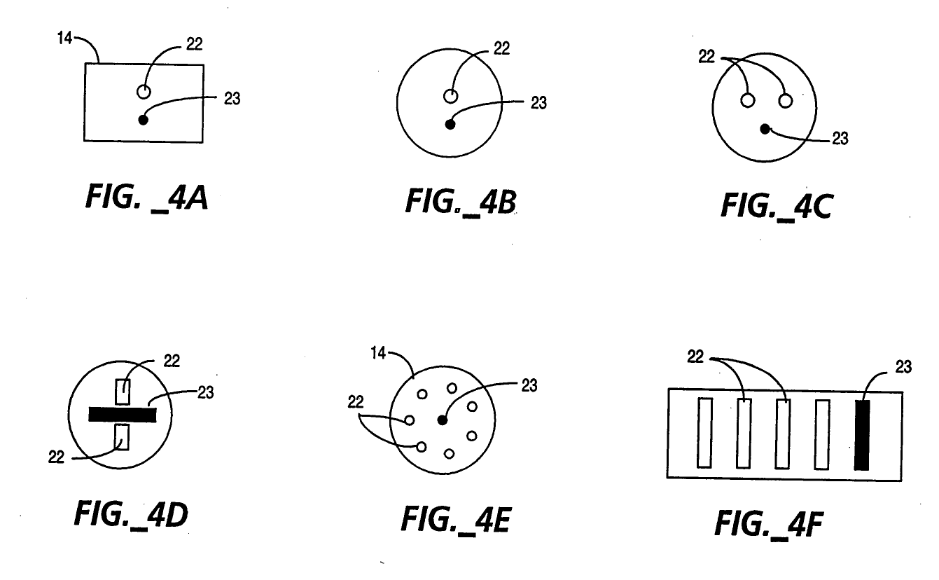

Briefly, referring to FIGS. 1 to 3 herein, the analytical device typically comprises a housing unit or top and bottom support members (10 & 16) which hold together and contain a reaction membrane (13) in fluid communication with an absorbent body (15). The top of the housing or top support member (10) has an open area or port (11) which exposes a portion of the upper surface (14) of the reaction membrane (13). During the performance of the assay, the liquid sample to be tested for the presence of a target substance is applied to the exposed surface of the reaction membrane. A limited region of the upper surface of the reaction membrane has a receptor adhered thereon to which the target substance, if present in the liquid sample, specifically binds. This limited region is referred to herein as the “receptor area” (22). The upper surface of the reaction membrane may optionally have a “control area” (23), which contains a control substance that, upon completion of the assay, indicates whether or not the assay was properly performed. Various types of control substances are known in the art. For example, the use of Protein A control regions are disclosed in U.S. Pat. No. 5,541,059. As known in the art, and as discussed in more detail in copending application Ser. No. 08/823,436, more than one type of receptor molecule can be immobilized onto the reaction membrane at multiple receptor areas to test for the presence of multiple target substances (i.e. analytes) in the sample. This is indicated in FIGS. 4c, 4e, and 4f, which show multiple receptor areas (22).

Any suitable porous material capable of immobilizing the receptor reagent employed in the analytical assay, can be used for the reaction membrane. Suitable materials include nitrocellulose, glass fiber, polyester, cellulose nitrate, polyester, polycarbon, nylon and other natural and synthetic materials which can be coupled directly or indirectly to the selected receptor. Generally, the reaction membrane is hydrophobic and partially hydrophilic and comprises partial positive and/or negative charges that allow the receptor molecule to bind. Certain membrane materials are charged, such as cellulose nitrate which has partial negative charges contributed by the nitro groups. Other materials may be pre-treated to provide a charged membrane. For example, polyester can be derivatized with carboxyl or amino groups to provide either a negatively or positively charged membrane. Nylon can be treated with acid to break peptide bonds to provide positive charges (from the amine groups) and negative charges (from the carboxyl groups). Porosity of the reaction membrane can also have a significant influence on the flow rate of the sample and assay sensitivity. For most assays, the porosity of the membrane is preferably in the range of about 0.1 to about 12 microns, and more preferably about 0.45 to 3 microns. U.S. application Ser. No. 08/823,936, discloses additional membrane properties that may influence assay results.

The term “reaction membrane” is intended to include the porous material to which the liquid sample is applied during the performance of the analytical assay, as well as additional porous supporting material, if any, that forms the lower surface of the reaction membrane. For example, a preferred reaction membrane comprises a sheet of nitrocellulose backed with a porous paper. Commercially available porous polyester supported nitrocellulose can also be used. A representative example of paper-backed nitrocellulose is commercially available from EY Laboratories Inc. (San Mateo, Calif.; Cat. Nos. PBNC15-1, PBNC15-10, PBNC15M-1, and PBNC15M-10). This preferred membrane is substantially more durable than nitrocellulose alone and can be employed without any other support component. This allows for easier handling and device assembly. This is presumably because when sample is added to the reaction membrane, it tends to flow more readily through the portions of the reaction membrane that have a high surfactant concentration as opposed to the more hydrophobic portions of the membrane. It will appreciate that the properties of a reaction membrane of a specified material can vary from lot to lot, and with age. Therefore, quality control testing, using standard controls, is performed in order to determine the suitability of a particular membrane for a given analytical assay.

When using a surfactant, the analytical devices are preferably assembled using reaction membranes that have not been blocked with protein-containing reagents. The term “blocked” is understood by those skilled in the art of membrane-based analytical assay design to refer to the treatment of a reaction membrane with a composition that prevents the non-specific binding of the target substance to the reaction membrane. Typically a blocking composition comprises a protein, such as casein or albumin, and may additionally comprise surfactants. The function of the protein is to bind to the reaction membrane to prevent the sample and/or assay reagents from binding non-specifically to the reaction membrane. Because the analytical assay devices of the present invention preferably do not employ blocked reaction membranes, and blocking steps are not required during the performance of the assay, the devices are more simple to manufacture than typical analytical assay devices, and the assays are easier to perform.

After the analytical devices are assembled, for example, using the methods detailed in U.S. Pat. No. 5,885,526, the reaction membrane may be prevented by treatment with a solution that contains surfactant, preferably in a high concentration. The phrase “treated with a surfactant”, means simply that a surfactant-containing solution, such as phosphated buffer saline solutions, has been applied to all or part of the exposed surface of the reaction membrane, and allowed to sufficiently dry prior to performing an analytical assay. Best results are usually achieved when the surfactant containing solution consists essentially of the surfactant and the solvent used to prepare the solution (e.g. water, alcohol, or other solvent). However, in some applications, it may be desirable to have other additional components included in the surfactant-containing solution. One or more receptor areas is also formed on the reaction membrane in a manner such that the resulting reaction membrane contains a higher concentration of surfactant at portions of the exposed surface of the reaction membrane where receptor areas are located relative to portions of the exposed surface of the reaction membrane that are peripheral to the receptor areas. The details of surfactant treatment are disclosed in U.S. Pat. No. 6,284,194 incorporated herein by reference.

When the reaction membrane is treated with certain surfactant-containing solutions having high concentrations of surfactant, greater than about 0.2 percent, and usually in the range of about 0.2 to about 15.0 percent (although higher concentrations can sometimes be used depending upon the solubility of the surfactant), increased flow of the sample and other reagents through the center of the reaction membrane where the receptor molecule is typically located, can be achieved. In typical membrane-based analytical assays, increased sample flow equates to a shorter reaction time between the target substance in the liquid sample and the receptor molecule located on the reaction membrane, and results in decreased assay sensitivity. However, with the analytical devices of the present invention, there is increased sample flow where the receptor area(s) is located, and reduced sample flow at portions of the membrane where there are no receptor areas, causing more sample to flow through the receptor area. Thus, the higher concentration of surfactant at the receptor area in effect, acts as a funnel that directs sample flow to the region of the membrane where receptor is located. This has the net effect of increasing assay sensitivity.

The surfactant treatment may be done by applying a surfactant-containing solution to the exposed surface of the reaction membrane of an already-assembled analytical device, in an amount sufficient so that all, or most of the exposed surface is contacted with the surfactant. Depending upon the surfactant used, the concentration of surfactant is typically in the range of about 0.2 to about 15.0%. The surfactant can be diluted in water, alcohols, or other suitable solvents (many commercial surfactants comprise proprietary solvent bases). Typically, about 20 to 50 μl surfactant-containing solution is used to treat a reaction membrane having an exposed surface area of 1 cm².

Preferred surfactants that achieve the above-described back-flow/funnel effect are anionic surfactants having molecular weights of less than about 1,000 which may be used alone or in combination with other surfactants. More preferably, the anionic surfactant used in the surfactant-containing solution has a molecular weight of less than about 800, and even more preferably, less than about 500. The surfactant-containing solution usually comprises at least 0.2% surfactant. Some anionic surfactants, such as sodium dodecyl sulfate (SDS), will work at lower concentrations. However, as the sensitivity achieved with SDS is generally low, it is not a preferred surfactant for some assays. A preferred surfactant-containing solution comprises from about 0.2% to about 2% of a cholic acid surfactant. When less than about 0.1% cholic acid surfactant is used to treat the membrane, sample flow decreases and sensitivity is reduced.

Alternatively, only the portion of the reaction membrane where receptor reagent(s) is (or will be) located, is treated with the surfactant-containing solution. With this embodiment of the invention, a somewhat higher concentration of surfactant is generally used compared to when the entire exposed surface of the reaction membrane is treated.

Typically, the glycoprotein sample is spotted, dropped, printed, or biojected onto the reaction membrane, using methods known in the art, so that the glycoprotein is adhered to a limited portion of reaction membrane. In the simplest embodiment of the invention, a drop of glycoprotein-containing composition is spotted onto the center of the reaction membrane so that a circular receptor area forms, as depicted in FIGS. 4a to 4c. For a circular receptor area having a diameter of approximately 1 to 4 mm on a nitrocellulose reaction membrane, approximately 0.5 to 2.5 μl of the receptor-containing composition is added to the center of the reaction membrane. Other methods can be used to achieve receptor areas having different shapes. For example, bar-shaped receptor areas, as depicted in FIG. 4d, can be used to form plus and minus signs as described in U.S. Pat. No. 4,916,056. Any other shapes of receptor areas can be used such as dots or stars.

After the glycoprotein sample is added to the reaction membrane, an appropriate detection reagent is added which specifically binds to the predetermined carbohydrate in the glycoprotein sample, if present. Prior to addition of the detection reagent, a wash buffer may be added to remove residual sample from the reaction membrane. However, in a preferred embodiment of the invention, the detection reagent is formulated in a detergent base that washes away residual sample. Thus, the wash and detection steps are combined into one, which simplifies the assay.

A preferred labeled lectin reagent is lectin/colloidal gold conjugate diluted in a detergent composition that is described in more detail below. The use of lectin/colloidal gold as a detection reagent is well-known in the art. Colloidal gold is a preferred label because colloidal gold conjugates are much more simple to prepare and use in comparison with conventional enzyme conjugate labels. Colloidal gold is purplish-red (or ruby-red) in color, and thus can be detected visually without the use of the instrumentation that is required for the detection of other types of markers such as radioactive isotopes, fluorescent markers, bioluminescent markers and chemiluminescent markers and other well known markers in analytical assays. Furthermore, unlike enzyme markers, colloidal gold particle markers do not require the additional step of adding a substrate. However, these other markers can be used within the scope of the invention.

Another type of labeling system is one in which a bridge is used between the sample and label to avoid steric hindrance. For example the lectin which binds the sample may be unlabeled and conjugated with biotin. Then an avidin-labeled conjugate is bound to the biotin on the lectin-biotin conjugate previously bound to the sample by flowing through the membrane. The roles of the biotin and avidin can be reversed. This approach is referred to as the indirect labeling approach.

A preferred diluent for the lectin/colloidal gold is a detergent-containing composition comprising one or more of the following detergents: TRITON® X-305, TRITON® X-100, TWEEN® 20, PLURONIC® L64, and BRIJ® 35. The TRITON® series of detergents are nonionic detergents comprising polyoxyethylene ethers and other surface-active compounds. The PLURONIC® series are nonionic surfactants that are partial esters of block copolymers of poly(oxyethene-co-oxypropylene). The TWEEN® series are derived from the SPAN® products by adding polyoxyethylene chains to the none-sterified hydroxyls. BRIJ® 35 is a trademark of the Pierce Chemical Company, Rockford, Ill., and is a 30% solution of polyoxyethylene lauryl ether detergent. Any combination of the above-listed detergents or other detergents or surfactants with similar properties can be used. Usually, the final concentration of detergent is in the range of from about 0.5% to about 3.0% detergent; about 1.0 to 1.5% detergent usually works best.

In some cases, a purplish-red color at the receptor area, indicating the presence of target substance, will be immediately apparent after addition of the colloidal gold to the reaction membrane, making a final wash step unnecessary if only qualitative results are desired (i.e. test results are either “positive” or “negative”). However, if quantitative results are desired, or if the presence of background “noise” (i.e. color on portions of the reaction membrane where the receptor reagent is not present) interferes with the reading of the receptor area, a final wash step can be employed using a water wash, or a detergent composition (which may be the same as or different from the diluent used to prepare the detection reagent). For quantitative results, the receptor area can be measured using any device designed for making such measurements, such as the optical analyzer described in U.S. Pat. No. 5,717,778.

All references, patents, and patent applications cited herein are hereby incorporated by reference in their entireties.

The following examples are for illustrative purposes only and are not to be construed as limiting the scope of the invention in any manner. In general, the examples illustrate:
A. How to make a rapid flow-through type of assay to identify carbohydrates using lectins.
B. The effect of the formation of the carbohydrate and lectin complex, pH, ionic strength, buffer species, etc.
C. Lectin and carbohydrate binding is very specific as demonstrated in direct assay procedure.
D. A demonstration of how to use this rapid assay technique to find if two same or different lectins will give cross reactivity because of a carbohydrate linked lectin.
E. When multiple lectins are used on the device membrane, a labeled glycoprotein's carbohydrate can quickly be identified by observing which lectin will give response in color. Then, the particular carbohydrate can be deduced.

In a sandwich assay, the sample avoids being colloidal gold labeled and, assuming the glycoprotein has more than one or different carbohydrates on the protein, a labeled lectin can add information of which carbohydrate this sample has when the right sandwich assay causes a bright spot on the device membrane. This tells one that the sample may have several kind of carbohydrates at one time when more than one bright spot is on the device membrane.

### EXAMPLES

### Example 1

### Assembly of Analytical Device

Analytical devices like those described in U.S. Pat. No. 5,885,526 were prepared using the following components and were assembled as shown in FIGS. 1-3: Top support layers (10) measuring 3.8 cm square were cut from flexible, but rigid polyvinyl chloride (PVC) plastic that had a water-insoluble pressure-sensitive adhesive on one side. Holes 8 mm in diameter were punched into the center of the top support layers. Circular reaction membranes (13), 11 mm in diameter, were punched from paper-backed nitrocellulose having a thickness of approximately 0.8 mm (EY Laboratories Inc. Cat. # PBNC15-1) and adhered to the adhesive side of the top support layer so as to cover the hole. An absorbent body (15) comprised of a 3.8 cm square of absorbent material (from Whatman, Cat. No. F427-05) was adhered to the adhesive side of the top support layer. A bottom support layer (16), measuring 3.8 cm square and comprising the same plastic material and adhesive as the top support layer, was adhered to the lower surface of the absorbent body. The same device described above can have an 8 mm diameter hole.

### Example 2

### Treatment of Reaction Membrane

A. Detergent Treatment of Entire Exposed Surface of Reaction Membrane

A 1% solution of sodium cholate was prepared in water. 40 μl of the cholate solution was added to the membrane of a pre-assembled analytical device prepared according to Example 1. The detergent solution completely covered the exposed upper surface of the reaction membrane and was allowed to absorb into the membrane. The membrane was allowed to dry over night at room temperature. After the membrane was completely dried, 0.5 μl of a solution containing 0.1-0.5 μg lectin in phosphate or Tris-glycine buffer was spotted onto the center of the reaction membrane and allowed to dry.

B. Detergent Treatment Limited to Receptor Area of Reaction Membrane

A 2.0% solution of sodium cholate is prepared in water. 0.5 μl of the sodium cholate solution is spotted onto the reaction membrane of a pre-assembled analytical device prepared according to Example 1, and allowed to dry for four hours at room temperature. 0.5 μl of a solution containing 100 ng glycoprotein antigen is spotted onto the reaction membrane at the same location where the detergent was spotted, and allowed to dry.

### Example 3

### Preparation of Reagents for Immunoassay

A. Colloidal Gold

Lyophilized lectin/colloidal gold is reconstituted in a wash buffer comprising the following surfactants diluted in 0.2M Tris: TRITON® X-305, TRITON® X-100 TWEEN® 20, PLURONIC® L64, and BRIJ® 35. Each detergent was used in amounts of 0.2% to achieve a final concentration of 1.0% detergent.

### Example 4

Stock phosphate buffer preparation is based on the methods described in the first chapter of Methods in Enzmmology, Vol. I (edited by Colowick, Kaplan). Buffer concentration is 100 mM. The same preparation procedure for Tris-glycine buffer and sodium bicarbonate buffer. Calcium chloride dihydrate solution 20 mM is prepared as stock solution. It is dissolved in distilled water.

A Tris-calcium buffer (TCB) is used. This buffer solution is composed of 100 mM Tris-glycine of desired pH taken from 2 mM calcium ion stock solution. 40 μl lectin—gold conjugates (stock solution, final OD520 nm reading about 2.0) is added.

Based on the above procedure, 40 μl of Con A—gold conjugate is diluted in each buffer at the desired pH.

All subsequent examples use this procedure with the diluted colloidal gold lectin conjugates unless it is otherwise specially instructed.

The assay procedures are as follows:

For direct detection, the membrane of a device as shown in the drawings is pre-inoculated with neoglycoprotein (10 mg/ml) or lectin (10 mg/ml) about 0.5-1.0 i11 and dried. The assay procedure for indirect detection (sandwich) preinoculated with lectin is as set forth above.

### Example 5

For this example, conjugates of avidin and biotin with lectins identified in the EY publication are used in the following protocol.

Reagent preparation:
1. Lyophylized biotinylated-lectin (“B-Con A”) is dissolved in Tris-glycine buffer pH 7.5, 50 mM with 2 mM calcium ions. 10 mg/ml is used as stock solution.
2. The B-Con A is diluted to 1 mg/ml using the same buffer of step 1. A solution of the lyophylized Con A—gold from the EY publication is dissolved in Tris-glycine buffer with final OD520 @ above 2/ml.

Assay Procedure: Direct Method.
1. 1 μl BM-BSA (branched mannose covalently linked to bovine serum albumin) is pre-inoculated at the center of a reaction membrane of the device described above and dried for 6 minutes under a 100V light bulb (or at room temperature overnight).
2. A solution of lectin gold conjugate flows through the membrane to form a sandwich.
3. The membrane is washed and the retained gold conjugate is read.
   Indirect Method.
1. The membrane is pre-inoculated with 1 ul Con A (10 mg/ml).
2. A sample of 40 ul BM-BSA (100 ug/ml) is added.
3. Biotinylated Con A—gold 40 ul flows through the membrane.
4. Streptavidin-gold (OD520=2. or above) 40 ul is added.
5. Wash with 80 ul washing solution and read the result by the aforementioned optical analyzer.

### Example 6

This example illustrates BM-BSA as a sample in a Con A/Con A-gold rapid assay in a dosage response study.

Fill 5 test tubes with 90 ul buffer Tris-glycine 100 mM pH 7.5 stock solution containing 2 mM calcium ion. To the first one, add 10 ul of M-BSA stock solution (10 mg/ml). Mix the content well in the tube. From this tube, take 10 ul to add to the next tube. Perform this in sequence so that you will have a series of five tubes each being diluted from the previous one 10×.

Procedure: A. Preparation of device for assay—the membrane is pre-inoculated with 1 ul of Con A and dried (Con A is in 2 mM borate buffer with 0.02% sodium azide). Assay procedure: 1. The membrane, then the surface is washed, is directed through the membrane, is pre-wet with 20 ul of buffer. 40 ul of diluted BM-BSA is directed through the membrane. 40 ul Con A-gold conjugate with buffer solution and the results are read by instrument in the following Table.

Con A/Con A - Gold Conjugate Dosage Response Curve
Concentration in each tube of BM-BSA in ug/ml

| | 10 | 1.0 | 0.1 | blank |
|---|---|---|---|---|
| CMR | 5.4 | 1.4 | 0.6 | −0.1 |
| Reading | 4.4 | 1.7 | 0.5 | −0.1 |

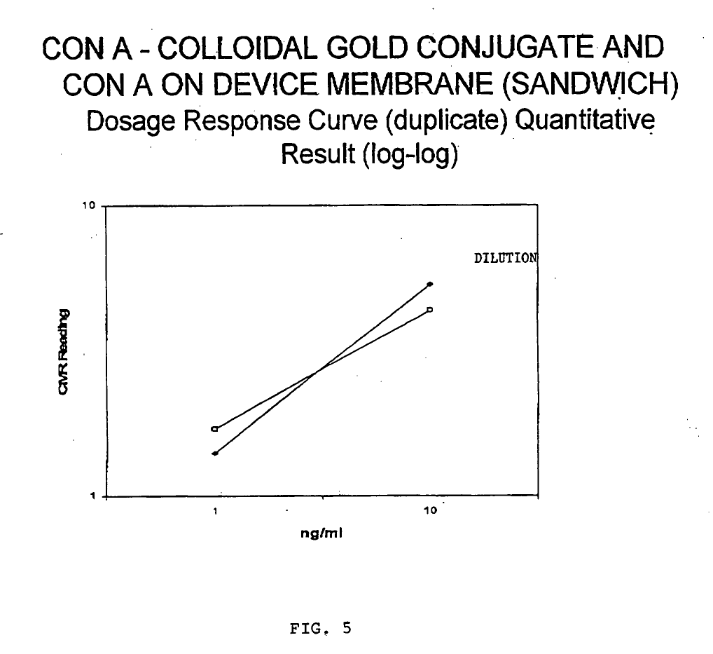

The data was used for the plot of FIG. 5.

### Example 7

In this example, the pH profile of mannose BSA and branched mannose-BSA are compared using the aforementioned optical instrument. The pH profile indicates the range of pH which should be used in a quantitative assay of about pH 6.4 to 7.5 in phosphate buffer to detect the indicated carbohydrates using Con A-colloidal gold conjugate. The results of these tests are illustrated in FIG. 6.

### Example 8

This example illustrates the effect of PEG on a carbohydrate/lectin rapid assay.

A mannose BSA sample is deposited onto the membrane. Then, a Con A-colloidal gold conjugate in solution flows through the membrane. In one instance, the surface is washed by flowing a PBS buffer solution through the membrane. This did not dissociate the specific binding. In another instance, a buffer solution supplied by EY Laboratories was used (including Tris glycine buffer, 2-6% PEG and 1.5% NaCl) was used. In this instance, the non-specifically bound label is washed. This illustrates the potential importance of testing for buffer solutions, even for washing.

### Example 9

This example illustrates a Fucose specific binding lectin cross-reactivity study in a direct detection procedure. Lotus and AAA specific bind with fucose BSA. UEA-1 binds non-specifically to other carbohydrates. The results of this test are shown in the following Table 1.

TABLE 1
Fucose Binding Lectin Cross-Reactivity
Lectin-Colloidal Gold Conjugate

| Lectin on Device | UEA-1 | Lotus | AAA |
|---|---|---|---|
| AAA | − | − | − |
| Lotus | − | − | − |

Since there is no cross-reactivity, these lectins are suitable for a sandwich assay. This type of testing and that of Example 10 for cross-reactivity serves to identify non-cross-reactive lectin reagent-receptor combinations for use in the invention.

### Example 10

In this example, another cross-reactivity study is shown. This shows that, as illustrated in the following Table 2.

This example shows that using BM-BSA as the glycoprotein, the sensitivity is better for LcH/DGL and DGL/DGL pair (see the first column result). The test shows whether there is cross-reaction between the same lectin or different lectin with same carbohydrate binding. In this study, the lectins are dissolved in Tris-glycine buffer containing 2 mM calcium divalent ion, pH 7.5. DGL, GNA/DGL and DGL/DGL and there is shadow on membrane when LcH/DGL pair is used. However, when the SBA-Gold is used, it cross-reacts with Con A, LcH and DGL and no binding at all with GNA. Con A has no galactose on it. So, most likely SBA is a glycoprotein with branched mannose on it. To substantiate this hypothesis, 1-10 ng/ml Bm-BSA is used as the glycoprotein, and it shows that the BM-BSA does not inhibit the interaction completely. When ug/ml BM-BSA is used, it inhibits cross-reaction except LcH.

This Table shows that SBA has low-cross reaction with HPA on a membrane or not in a reverse position. However, HPA has strong cross-reactivity with lotus. Thus, when a sandwich assay is used to capture a fucose-linked glycoprotein, AAA lectin may be used on the membrane because it has no cross-reactivity with SBA, GS-1, HPA and WGA. On the other hand, HPA is less preferred because it gives a shadow. Lectins such as HPA which yield a shadow can be used provided quantitative measurement is also used. There, the identification of a positive is indicated by a quantitative value above the cutoff level of the shadow.

In this study, the following conclusions are drawn: SBA on a membrane can pair with Lotus, AAA and SBA colloidal gold conjugates.

It cannot pair with HPA due to small cross-reactivity when in reverse HPA on a membrane and SBA is colloidal gold conjugates.

GS I is a lectin and will bind to Galactose and link to the next carbohydrate. It can pair only with AAA and SBA.

HPA has no cross-reactivity with AAA and SBA when HPA gold conjugate is used AAA and SBA on the device membrane. But, it is not recommended for use in reverse. WGA can pair with Lotus, AAA and SBA. It will give strong positive response to HPA, but can be washed away.

The IND in the Table for WGA indicates that before washing the WGA and HPA cross-react to provide a strong dot. However, after washing the dot appears indicating weak binding.

TABLE 2
Lectin Cross-Reactivity Study
Lectin-Colloidal Gold Conjugate

| Lectin on Device | SBA | GS-1 | HPA | WGA |
|---|---|---|---|---|
| Lotus | − | Sh | + | − |
| AAA | − | − | − | − |
| SBA | − | − | − | − |
| HPA | Sh+ | Sh+ | Sh− | IND |

Sh = Shadow
IND = Indeterminant

### Example 11

This is a check board study using a sandwich assay method which is a more sensitive method for detecting the glycoprotein than with the direct method. The performance characteristics of four different lectin on the mannose specific binding property tested for galactose specific binding, using DGL-Gold conjugates and SBA. The results are shown in the following Table 3.

TABLE 3
Reactivity Study of Branched Mannose-BAS Using Lectin and
Lectin Gold Conjugates in a Sandwich Format
Lectin Gold Conjugate

| Lectin on Device | DGL | Blank | SBA | SBA/BM (ng) | SBA/BM (μg) |
|---|---|---|---|---|---|
| CON A | + | − | + | W+ | − |
| LcH | ++ | Sh | + | W+ | + |
| GNA | W+ | − | − | − | − |
| DGL | ++ | − | + | + | − |

BM = Branched Mannose
W = Weak
Sh = Shadow

The left column on the checker board shows all mannose binding lectins. On the top, across the Table, shows colloidal gold conjugates to DGL, SBA. The blank means no mannose BSA is used in a sandwich assay. The SBA gold conjugate at first has no BM-BSA included in the conjugates while the subsequent column, the SBA/BM has ng concentration of BM in the gold conjugate and in the last column the BM concentration is increased to ug.

Thus, in a sandwich assay, BM is the sample, mannose specific lectin on the device membrane and the lectin gold conjugates (DGL-gold). The first column shows positive for all combinations with different mannose specific lectin on device membrane in a flow-through format.

In the blank column, there is only shadow between DGL and LcH. All others show none. Thus, there is no cross-reactivity of DGL with lectin in this example except LcH.

This means DGL can combine with Con A, GNA or itself in a sandwich assay and do not have to worry about the cross-reactivity.

But, when SBA is used without BM as sample, it shows cross-reactivity with all lectin on the left column except GNA.

One question is whether cross-reactivity is due to BM or Galactose on lectin molecules. It is not likely to be a terminal mannose, because there is no reaction between GNA and SBA gold.

We can assume that it is not galactose. This is because it is known that Con A is not a glycoprotein and it reacts with SBA. So, logically it might be a branched mannose.

To prove this point, branched mannose BSA is added at ng quantity in assay. It weakens the binding between Con A and LcH with SBA. When the concentration of BM increases to ug quantity, there is no reaction between Con A and SBA, DGL and SBA. This is because the BM blocked the binding of Con A and DGL at higher concentration. The reason why the LcH and SBA are still cross-reacted. This may be due to a a-Fucose branched out from BM extended oligosaccharides as known in literature gives a better binding than BM.

LcH, in other words, binds better with a BM having a-Fucose, which may be the sugar on SBA. So, BM by itself does not appear to block the cross-reactivity.

Conclusion—SBA has mannose or branched mannose on it. Most likely, it is not a single mannose and it is a branched. This is because GNA does not bind to SBA in the reaction. This approach illustrates testing to determine non-cross-reactive lectins for a sandwich assay.

### Example 12

In this example, the binding affinity of mannose BSA to lectin of the same binding specificity is compared and also with galactose BSA (GAL).

The results were illustrated in Table 4 which demonstrates the sensitivity and specificity of a lectin can be increased as shown in this case by GNA lectin.

TABLE 4
Mannose Binding Lectin Comparison
Glycoprotein Colloidal Gold Conjugate

| Lectin | M | BM | GAL | Fetuin | Asialofetuin |
|---|---|---|---|---|---|
| Con A | Sh | + | − | Sh | − |
| DGL | + | + | − | Sh | Sh |
| GNA 1× | − | Sh | − | − | Sh |
| GNA 4× | − | Sh | − | − | Sh |
| GNA Special | Sh+ | W+ | − | − | + |

M = Mannose
BM = Branched Mannose
W = Weak
Sh = Shadow

In this Table, in the left column, all lectin, Con A, DGL and GNA all will bind to mannose or branched mannose.

In this form of checker board, all lectins are inoculated on the device membrane.

All neoglycoprotein, M or BM or GAL (linked to BSA) and fetuin and asialofetuin are colloidal gold conjugates.

Interpretation of the Results

Across the first line, the Con A as expected should bind to M and BM (mannose or branched mannose linked BSA), not with galactose. The only difference between mannose and galactose on the carbohydrate structure skeleton is that mannose has hydroxyl group at carbon 4 at equitorial while galactose has the same hydroxyl group at vertical position.

This is a very small difference, but the lectin can differentiate them in less than 1 minute rapid assay.

The second lectin OGL in the left column shows strong binding to both M and BM. Apparently, this lectin has stronger binding constant with M and BM, but shows no reaction with galactose linked BSA.

Regarding the glycoprotein fetuin and asialofetuin, both lectins bind weakly or no binding because of steric hindrance. One of the speculations is that mannose may be facing the colloidal gold so not available for binding.

When GNA is used, from increasing GNA on device membrane by 4×, the reaction of binding does not increase. However, when the colloidal gold conjugated of GNA is in a solution containing 4-6% PEG in Tris-glycine buffer, the binding increases with M, BM and strongly with asialofetuin. It is well known, in ELISA, PEG increases immunocomplex formation. In this case, the environment of reaction condition helps to increase binding activity.

Lectin with same carbohydrate binding specificity do not bind with the same strength. That is, the DGL is better than Con A and better than GNA. The binding affinity constant can be increased with the environment where the binding takes place.

### Example 13

This example illustrates lectin carbohydrate specificity in a rapid flow-though assay.

The results are shown in Table 5.

TABLE 5
Lectin Carbohydrate Specificity: Practical Example in a Rapid Assay
Lectin-Colloidal Gold Conjugate

| Neoglycoprotein | Con-A | SBA | WGA | AAA | HPA |
|---|---|---|---|---|---|
| M-BSA | W+ | − | − | − | − |
| BM-BSA | + | − | − | − | − |
| GAL-BSA | − | + | − | − | Sh |
| GlcNAc-BSA | − | − | + | − | + |
| Fu-BSA | − | − | − | + | Sh |
| GalNAc-BSA | − | + | − | − | + |

M = Mannose
BM = Branched Mannose
W = Weak
Sh = Shadow

A. Under the designed experimental parameter, the pH, the ionic strength, the selected buffer species and washing solution, the lectin will perform to demonstrate the specific carbohydrate binding. You have 6 neoglycoprotein inoculated at 6 different device membrane, Con A will pick only those with mannose, SBA binds only those with Galactose BSA and GalNAc linked BSA, and so forth. This illustrates carbohydrate identification.
B. Differentiation using 2 lectins. When the unknown glycoprotein binds to WGA and HPA, you will know that you may have GalNAc and/or GlcNAc. When you use SBA, if it binds, you can be sure the unknown has both.

A detailed explanation of the results is as follows.

In this Table, on the left, is a column of neoglycoprqtein (that is, the monosaccharides (MonoS)) is covalently linked to the Bovine Serum Albumin (BSA). For example, M-BSA, only mannose is linked onto the BSA. BM means branched mannose. GAL-BSA means galactose linked BSA. GlcNAc-BSA means N-acetyl Glucosamine, Fu-BSA means Fucosylated BSA and GalNAc-BSA is N-acetyl Galactosime-BSA. All of these are selectively inoculated on each device. Across the top, there are five different lectins, all colloidal gold conjugated.

In a rapid direct assay procedure and the checker board format, one can in a few minutes demonstrate the specific binding of each lectin.

Interpretation of the Result

Con A is known to bind to mannose and branched mannose. So, the result shows exactly the weak binding with M-BSA (light dot) and strong with branched mannose BSA with strong red dot.

Con A also shows non-reactive with all other four neoglycoprotein.

SBA is known to react with Galactose and GalNAc. This checker board shows exactly what it binds and this lectin is non-reactive with mannose, or GlcNAc, or a-Fucose linked BSA. This lectin is purified from Soy Bean.

WGA, a lectin from Wheat Germ, in this checker board study, binds only with N-acetyl-Glucosamine (GlcNac) or sialic acid. It does not react with other monosaccarides.

AAA is a lectin from eel. In this study, it only binds with a-Fucose linked BSA.

HPA is a lectin purified from snails from France. It has a broader carbohydrate reactivity. Strong positive with GalNAc and GlcNAc as shown on the device membrane by a bright dot. The binding with Galactose and a-Fucose is weak.

This study demonstrates that the rapid assay result can match the binding specificity of each lectin with known neoglycoprotein.

### Example 14

This example illustrates the specificity of a-Fucose binding reactivity.

TABLE 6
Fucose Binding Lectin Comparison
Lectin-Colloidal Gold Conjugate

| Oligosaccharide | UEA-1 | Lotus | AAA |
|---|---|---|---|
| Fu-BSA | + | + | + |
| M | Sh | − | − |
| BM | − | − | − |
| Gal | + | − | − |
| Glc | W+ | − | − |
| GlcNAc | − | − | − |
| GalNAc | − | − | − |
| Gal β (1-3) GlcNAc | W+ | − | − |
| Gal Nac β (1-4) Gal | − | − | − |

M = Mannose
BM = Branched Mannose
W = Weak
Sh = Shadow

In this study, Lotus and AAA colloidal gold labeled would bind only with a-Fucose linked BSA.

These two lectins show no binding to other neoglycoprotein.

On the contrary, UEA-I binds to different neoglycoproteins. This lectin was used for typing blood type (0). This means that this lectin binds to a-Fucose likely in a larger binding area of binding than one monosaccharide.

### Example 15

In this example, the method of Example 14 is used. The results are summarized in the following Table 7.

TABLE 7
Practical Applications in Glycobiology; Lectins for Carbohydrate
Identification and Differentiation
Identification of Unknown Carbohydrate Moiety on a Glycoprotein
Lectin-Colloidal Gold Conjugate

| Glycoprotein | Con A | GNA | SBA | Lotus | WGA |
|---|---|---|---|---|---|
| Fetuin | + | − | ++ | W+ | + |
| Asialofetuin | W+ | Sh | ++ | + | + |
| Lactoferrin | ++ | ++ | ++ | ++ | ++ |

M = Mannose
BM = Branched Mannose
W = Weak
Sh = Shadow

In this case, three native glycoproteins, fetuin, asialofetuin and lactoferrin, are used. In this Table, the positive response of Con A tells that all glycoproteins have branched mannose, GNA informs us fetuin has no terminal mannose. Asialofetuin may have a small amount. There could be a large amount of terminal mannose exposed for binding from lactoferrin. SBA shows that all three glycoproteins have galactose or GalNAc. All three glycoproteins have a-Fucose and GlcNAc because all gave positive response to the Lotus and WGA.

Instead of using five different devices, one can use one device membrane inoculated with five different lectins for five different monosaccarides, one of the three samples can be added, and then comes in a colloidal gold labeled lectin. Or, colloidal gold labeled one of the glycoprotein use it in a direct method to detect which lectin it would bind. As a result, one can deduce which carbohydrate sample has in minutes.

1. A method for rapid detection of at least a first carbohydrate in a carbohydrate-containing sample molecule comprising the steps of:
(a) retaining said sample molecule on a region of one liquid permeable reaction membrane,
(b) flowing a solution first lectin, capable of binding said first carbohydrate, through said one reaction membrane to selectively bind said first lectin to said retained first carbohydrate, said lectin being directly conjugated to a label prior to binding to the carbohydrate or being bound to a label on a separate molecule after binding of said first lectin to said carbohydrate,
(c) thereafter, detecting said label bound to said one reaction membrane, indicating the presence of said first carbohydrate.

2. The method of claim 1 in which said reaction membrane includes an immobilized receptor capable of binding said sample molecule, and said sample molecule is retained on said reaction membrane by binding to said immobilized receptor.

3. The method of claim 2 in which said immobilized receptor comprises a second lectin of the same type as, or a different type from, said first lectin.

4. The method of claim 3 in which said second lectin is a different type from said first lectin.

5. The method of claim 1 in which said reaction membrane is washed between steps (b) and (c).

6. The method of claim 1 in which said detection is quantitatively performed using an optical analyzer.

7. The method of claim 1 in which said sample molecule is retained on at a second discrete region of said one reaction membrane of a second liquid permeable reaction membrane and the procedure of claim 1 is repeated using at least a labeled second lectin of a different type than said first lectin and capable of selectively binding a second carbohydrate of said sample molecule.

8. The method of claim 1 performed in less than about 1 hour.

9. The method of claim 1 in which said separate molecule comprises avidin and said first lectin is conjugated to biotin.

10. The method of claim 1 in which said separate molecule comprises biotin and said first lectin is conjugated to avidin.
